(19) United States
(12) Patent Application Publication
(10) Pub. No.: US 2014/0106356 A1
(43) Pub. Date: Apr. 17, 2014

(54) KIT FOR DRUG METABOLISM DETERMINATION AND TOXICITY PREDICTION

(71) Applicant: NATIONAL TAIWAN UNIVERSITY, Taipei City (TW)

(72) Inventors: Hsuan-Shu Lee, Taipei City (TW); Guan-Tarn Huang, Taipei City (TW); Kai-Chiang Yang, Taipei City (TW); Tsai-Shin Chiang, Taipei City (TW); Feng-Huei Lin, Taipei City (TW)

(73) Assignee: NATIONAL TAIWAN UNIVERSITY, Taipei City (TW)

(21) Appl. No.: 13/650,634

(22) Filed: Oct. 12, 2012

(51) Int. Cl. C12N 5/10; C12Q 1/68

(52) U.S. Cl. 435/6.12; 435/325; 435/366; 435/397; 435/398; 435/396; 435/6.1; 435/6.17

(57) ABSTRACT

The present invention provides a kit comprising a cell transfected with hepatic transcription regulators, and a culture medium that support growth of the cell. The present invention further provides a method for determining drug metabolism and predicting drug toxicity, comprising transfecting a cell with hepatic transcription regulators, and culturing the cell on a medium.

### FIELD OF THE INVENTION

The present invention is related to a kit for drug metabolism determination and toxicity prediction and the methods used thereof.

### BACKGROUND OF THE INVENTION

The concern of drug safety is a serious issue for the public and health organizations, while a critical challenge to the pharmaceutical industry. Many drugs have been withdrawn from the market, and over 40% of marketing candidate drugs terminated due to unexpected toxic effects. This outcome represented a severe harm to the patients and a huge loss of money from the industry. For decades, drug-induced hepatotoxicity accounted for around 30% of drug withdrawals from the market. Apparently the traditionally used toxicity assays based on animal studies have failed to recognize the potential human risk of hepatotoxicity of these compounds during their development. Animal tests are expensive and of low throughput, yet frequently of questionable relevance to predicting human risks because of inter-species differences. Therefore, several government authorities have proposed an urgent requirement for moving away from traditional animal toxicology tests to new approaches based on well-designed in vitro platforms using human cells or tissues (Committee on Toxicity Testing and Assessment of Environment Agents, National Research Council. Toxicity testing in the 21st century: a vision and a strategy, 2007).

For in vitro evaluations of hepatotoxic risks, human liver slices, liver microsomes, hepatoma cell lines, and primary hepatocytes are used frequently. However, liver slices are viable for ˜1 day only, and human liver microsomes lack dynamic gene expression profiles. In spite of human hepatoma cell lines are relatively accessible, the expression levels and activities of drug-metabolizing enzymes have been shown extremely low, comparing to primary hepatocytes, beyond the range able to demonstrate certain drug-induced hepatotoxicity. Although human hepatocytes are the most ideal cells for predicting drug toxicity, the cell source is quite limited and it is difficult to propagate hepatocytes in culture dish. Furthermore, the activities of phase I cytochrome P450 (CYP) enzymes in hepatocytes decline rapidly under traditional culture conditions. Therefore, it is of critical importance to develop an in vitro platform with appropriate CYPs activities for risk assessment of drug-induced hepatotoxicity.

CYPs located in mitochondria and endoplasmic reticulum are the major phase I enzymes present in the liver for metabolizing endogenous and exogenous chemicals including drugs. CYP1, CYP2, and CYP3 family members are relevant to drug biotransformation in human liver. More than half of clinical drugs and xenobiotics in use today are metabolized by CYP3A4 and CYP2C9. However, their expression levels are usually very low in cells of currently used prediction systems. Expressions of these enzymes are coordinately regulated by several hepatocyte enriched transcription factors and nuclear receptors at transcriptional level in the liver. Besides, cell-cell communication and microenvironment stimulation also help the cultured hepatocytes to maintain their functions of these CYP enzymes.

Three dimensional (3D) culture systems have been shown to improve cell-cell and cell-matrix interactions and hence have positive effects to preserve structural polarity and maintain cellular functions. Over the last decade, many studies have demonstrated that higher levels of liver-specific functions of hepatocytes could be maintained better in 3D than in conventional monolayer culture. To this end, several 3D culturing methods such as spheroid formation, collagen sandwich gel culture, microencapsulation of cells, and seeding of cells into natural or synthetic biodegradable scaffolds have been commonly used for culturing hepatocytes and hepatic cell lines.

### BRIEF DESCRIPTION OF THE DRAWINGS

FIG. 1 shows the selection of essential genes optimally inducing CYP3A4 expression in HDFs by Q-PCR.

FIG. 2 shows the comparisons of 10F-HDF with 3 hepatoma cell lines Hep3B, flepG2 and HuH-7 regarding their expression levels (A) and enzyme activities (B) of CYP3A4, CYP1B1 and CYP2C9.

FIG. 3 shows the characterization of spheroids of 10F-HDFs formed on HydroCell dishes.

FIG. 4 shows the enhancement of CYP3A4 expression in 10F-HDFs cultured in various kinds of scaffolds.

FIG. 5 shows the enhancement of CYP gene expression (A) and enzyme activities (B) of 10F-HDFs in 3D cultures.

FIG. 6 shows the images of SEM (A-D) and confocal microscopy (E-F) of tri-copolymer scaffolds seeded with 10F-HDFs.

FIG. 7 shows the expression level (A) and enzyme activities (B) of CYP3A4 by the spheroid scaffold constructs of 10F-HDFs cultured for 1-4 weeks.

FIG. 8 shows the enhancement of CYP enzyme activities in HDFs, 2 MSCs and 2 ADSCs by 10-factor delivery and further 3D culturing into spheroid and spheroid-scaffold. (A) CYP3A4 enzyme activities, (B) CYP2C9 enzyme activities, and (C) CYP1B1 enzyme activities. #: indicates a significant difference between normal control in HDF and in other cell types (p<0.05). HDF=human dermal fibroblasts; MSC=mesenchymal stem cells; ADSC=adipose-derived stromal cells.

### SUMMARY OF THE INVENTION

The present invention provides a kit for drug metabolism determination and toxicity prediction and the methods used thereof, comprising a cell transfected with hepatic transcription regulators and a culture medium that support growth of the cell.

### DETAIL DESCRIPTION OF THE INVENTION

### Definitions

Unless otherwise specified, “a” or “an” means “one or more”.

As used herein, “hepatic transcription regulators” means hepatic transcription factors or nuclear receptors.

Compared to the limited source and the difficulty of propagating human hepatocyte, it is easier to obtain and propagate human dermal fibroblasts (HDF), mesenchymal stem cells (MSC), and adipose-derived stem cells (ADSC) in culture dish for the use in generating a platform for drug metabolism and toxicity tests. In this invention, a strategy to induce expression and activities of CYP3A4, CYP1B1 and CYP2C9 in HDF, MSC, and ADSC was established by delivery of several hepatocyte-enriched transcription factors and nuclear receptors using a lentivirus system. Next, the CYP activities were further augmented by forming the cells into spheroids and by sequential spheroid formation and scaffold cultivation in gelatin-chondroitin-hyaluronan (GCH) tricopolymer scaffolds (Gelatin-chondroitin-hyaluronan tri-copolymer scaffold for cartilage tissue engineering. Biomaterials 2003; 24:4853-8).

CYPs are major phase 1 enzymes playing central roles in the metabolism and detoxification of various endogenous and exogenous compounds introduced into the body. CYP3A4 is abundant in human liver and metabolize more than 50% of clinically used drugs. Therefore, in this invention CYP3A4 was used as the primary end to select factors in combination mostly enhancing its expression. It has been shown that liver-enriched transcription factors and nuclear receptors regulate expression of most CYPs at transcriptional level.

Therefore, the present invention provides a kit comprising:
(a) a cell transfected with hepatic transcription regulators; and
(b) a culture medium that support growth of the cell,
wherein the culture medium is selected from a 2-dimensional culture medium or a 3-dimensional scaffold.

According to the invention, the hepatic transcription regulators are selected from the group consisting of hepatocyte nuclear factor (Hnf) 1α, Hnf 1β, Hnf 4α, Hnf 6, aryl hydrocarbon receptor nuclear translocator (Arnt), pregnane X receptor (Pxr), GATA binding protein 6 (Gata6), interleukin 6 signal transducer (Gp130r), glucocorticoid receptor (Gr), and H2.0-like homeobox-1 (Hlx-1).

In one embodiment of the present invention, the cell is selected from a human dermal fibroblast, a mesenchymal stem cell, or an adipose-derived stem cell.

According to the invention, the cell forms monolayer or spheroid on the culture medium. In the preferred embodiment, the 3-dimensional scaffold is selected from a gelatin scaffold, a type I collagen scaffold, a type IV collagen scaffold, a keratin scaffold, or a gelatin-chondroitin-hyaluronan tri-copolymer scaffold. Other 3-dimensional scaffolds that are capable for improving cell growth can also be used.

According to the invention, the cell is induced to express cytochrome P450 (CYP) enzymes by the hepatic transcription regulators. The cytochrome P450 is selected from CYP3A4, CYP1B1 or CYP2C9.

The present invention further provides a method for determining a drug metabolism, comprising:
(a) transfecting a cell with hepatic transcription regulators;
(b) culturing the cell on a medium;
(c) adding the drug into the medium; and
(d) measuring a concentration change of the drug.

According to the invention, the hepatic transcription regulators are selected from the group consisting of hepatocyte nuclear factor (Hnf) 1α, Hnf 1β, Hnf 4α, Hnf 6, aryl hydrocarbon receptor nuclear translocator (Arnt), pregnane X receptor (Pxr), GATA binding protein 6 (Gata6), interleukin 6 signal transducer (Gp130r), glucocorticoid receptor (Gr), and H2.0-like homeobox-1 (Hlx-1).

In one embodiment of the present invention, the cell is selected from a human dermal fibroblast, a mesenchymal stem cell, or an adipose-derived stem cell.

According to the invention, the medium is selected from a 2-dimensional culture medium or a 3-dimensional scaffold and the cell forms monolayer or spheroid on the medium. In the preferred embodiment, the 3-dimensional scaffold is selected from a gelatin scaffold, a type I collagen scaffold, a type IV collagen scaffold, a keratin scaffold, or a gelatin-chondroitin-hyaluronan tri-copolymer scaffold. Other 3-dimensional scaffolds that are capable for improving cell growth can also be used.

According to the invention, the cell is induced to express cytochrome P450 (CYP) enzymes by the hepatic transcription regulators. The cytochrome P450 is selected from CYP3A4, CYP1B1 or CYP2C9.

The present invention also provides a method for predicting a drug toxicity, comprising:
(a) transfecting a cell with hepatic transcription regulators;
(b) culturing the cell on a medium;
(c) adding the drug into the medium; and
(d) measuring a death rate of the cell.

According to the invention, the hepatic transcription regulators are selected from the group consisting of hepatocyte nuclear factor (Hnf) 1α, Hnf 1β, Hnf 4α, Hnf 6, aryl hydrocarbon receptor nuclear translocator (Arnt), pregnane X receptor (Pxr), GATA binding protein 6 (Gata6), interleukin 6 signal transducer (Gp130r), glucocorticoid receptor (Gr), and H2.0-like homeobox-1 (Hlx-1).

In one embodiment of the present invention, the cell death rate can be measured by lactate dehydrogenase (LDH)-cytotoxicity assay.

In one embodiment of the present invention, the cell is selected from a human dermal fibroblast, a mesenchymal stem cell, or an adipose-derived stem cell.

According to the invention, the medium is selected from a 2-dimensional culture medium or a 3-dimensional scaffold and the cell forms monolayer or spheroid on the medium. In the preferred embodiment, the 3-dimensional scaffold is selected from a gelatin scaffold, a type I collagen scaffold, a type IV collagen scaffold, a keratin scaffold, or a gelatin-chondroitin-hyaluronan tri-copolymer scaffold. Other 3-dimensional scaffolds that are capable for improving cell growth can also be used.

According to the invention, the cell is induced to express cytochrome P450 (CYP) enzymes by the hepatic transcription regulators. The cytochrome P450 is selected from CYP3A4, CYP1B1 or CYP2C9.

### EXAMPLES

The examples below are non-limiting and are merely representative of various aspects and features of the present invention.

### Cell Culture

Retrieval and use of human tissue and cells were approved by the Research Ethical Committee at NTUH (201201007RID). HDFs were primarily cultured from human foreskin tissues obtained by circumcision. The human embryonic kidney 293T cells and human hepatoma cell lines HepG2 and Hep3B were obtained from American Type Culture Collection (ATCC, Manassas, Va., USA). Another human hepatoma cell line HuH-7 was from Japanese Collection of Research Bioresources (JCRB, Ibaraki, Osaka, Japan). All the cells were cultured in Dulbecco's Modified Eagle's Medium (DMEM, GIBCO-BRL, Scotland, UK) supplemented with 10% fetal bovine serum (FBS) at 37° C. under an atmosphere containing 5% CO2. HDFs between 7 and 10 passages were used in this invention. To be formed into spheroids, HDFs were cultured on HydroCell dishes (Thermo Scientific, Tokyo, Japan) whose surface was covalently immobilized with super hydrophilic polymer to prevent cell attachment.

### Plasmid Construction and Production of Recombinant Lentiviruses

The coding sequence of the complementary DNAs (cDNAs) of 24 human hepatocyte-enriched transcription factors and nuclear receptors were either cloned by reverse transcription-polymerase chain reaction (RT-PCR) from human hepatoma cell lines or directly synthesized. These cDNAs were subcloned into pCDH cDNA Expression Lentivector (System Biosciences, Mountain View, Calif., USA). The expression of the inserts would be driven by a CMV promoter while a reporter copGFP be driven by a separate promoter EF1. The cloned genes include hepatocyte nuclear factor (Hnf)1α, Hnf1β, Hnf3α, Hnf3β, Hnf4α, Hnf6, CCAAT-enhancer binding protein alpha (C/ebpα) and beta (C/ebpβ), GATA binding protein 6 (Gata6), V-rel reticuloendotheliosis viral oncogene homolog A (Rel-a), SRY-box (Sox17), H2.0-like homeobox-1 (Hlx-1), X-box-binding protein 1 (Xbp-1), aryl hydrocarbon receptor (AhR), pregnane X receptor (Pxr), retinoid X receptor (Rxr), liver X receptor (Lxr), farnesoid X receptor (Fxr), constitutive androstane receptor (Car), nuclear receptor subfamily 2, group F, member 2 (Nr2f2), nuclear receptor subfamily 5, group A, member 2 (Nr5a2), aryl hydrocarbon receptor nuclear translocator (Arnt), glucocorticoid receptor (Gr), and interleukin 6 signal transducer (Gp130r). To produce recombinant lentiviruses, 293T cells (1×10⁶ cells) were seeded into 10-cm dishes and co-transfected with respective recombinant expression lentivectors in combination with envelop plasmid pMD2.G and packaging plasmid psPAX2 (deposited to Addgene, Cambridge, Mass., by Dr. Didier Trono, School of Life Sciences and Frontiers in Genetics Program, Lausanne, Switzerland) using jetPEI™ transfection reagent (Polyplus-Transfection Inc., New York, N.Y., USA) in accordance with manufacturer's instructions. Supernatants from the transfected 293T cells were collected 2 days after transfection and concentrated by centrifugation (4000×g for 30-60 min at 4° C.) using Amicon Ultra 15 Centrifugal Filter Units (Millpore, Billerica, Mass., USA). After filtered through 0.2 μm cellulose acetate filters, virus solutions were stocked in aliquots at −80° C. until used.

### Fabrication of Scaffolds

The scaffolds were produced as previously described with some modifications (Gelatin-chondroitin-hyaluronan tri-copolymer scaffold for cartilage tissue engineering. Biomaterials 2003; 24:4853-8: Bone marrow combined with dental bud cells promotes tooth regeneration in miniature pig model. Artif Organs 2011; 35:113-21). In brief, gelatin (Sigma-Aldrich), chondroitin-6-sulfate (Sigma-Aldrich), sodium hyaluronate (Seikagaku Corp., Tokyo, Japan), type I collagen (BD Biosciences, Bedford, Mass., USA) and type IV collagen (NittaGelatin Inc., Osaka, Japan) were purchased and keratin was homemade from human hair bleached by peracetaic acid and protein extracted in 100 mM Tris. Respective materials or mixture of gelatin, chondroitin and hyaluronan (for producing GCH tri-copolymer scaffold) were cross-linked in 0.1% glutaraldehyde (Sigma-Aldrich), frozen overnight, lyophilized, cross-linked again by glutaraldehyde, treated by 0.1 M glycine to remove residual free glutaraldehyde, and at last lyophilized again. The fabricated gelatin, GCH tri-copolymer, type I and type IV collagen and keratin scaffolds were now ready for use. These scaffolds were cut into 3×3×3 mm cubes for cell seeding. The pore size of scaffolds thus formed was in the range between 150 and 200 μm and the porosity around 75-80%.

### Statistics

Data were presented as mean±SE. A one-way ANOVA analysis with a post-hoc Dunnett's multiple comparison tests was used to analyze the differences between subgroups. Differences were considered statistically significant at P values less than 0.05. For all statistics, data from triplicate or 3 independent experiments were used.

### Example 1

### Selection of 10 from 24 Factors Optimally Inducing CYP3A4 Expression in HDFs

Because CYP3A4 is the most abundant and important among liver CYPs in metabolizing exogenous xenobiotics and drugs, the selection of a combination among the 24 cloned hepatocyte specific factors to optimally express CYP3A4 in HDFs was conducted. The recombinant lentiviruses (each at 75 MOI) were used to infect HDFs for 2 weeks in ordinary culture dishes coated with polystyrene. Aliquots of 1×10⁴ HDFs in 0.5 mL of culture medium were seeded in 24-well plates and cultured for 1 day before they were infected by indicated combinations and multiplicity of infection (MOI) of recombinant lentiviruses. The virus-infected cells were maintained for 2 weeks in 24-well plates with a medium change every 2-3 days. In addition to all the prepared 24 factors, the infection of the cells using virus pools omitting respective one from the 24 factors were also tested.

Total RNAs of the cells were extracted using REzol^{IM} C&T reagent (Protech Technologies Inc., Taipei, Taiwan). Reverse transcription was performed by Super-Script III reverse transcriptase (Invitrogen, Carlsbad, Calif., USA) with a total volume of 20 L and the products were used for Q-PCR in which TaqMan system (Applied Biosystems, Foster City, Calif., USA) with respective primers/probes was used for chain reaction referring to a house-keeping gene glyceraldehyde-3-phosphate dehydrogenase (GAPDH). The PCR conditions were denaturizing at 95° C. for 30 s, annealing at 60° C. for 30 s, and extension at 72° C. for 30 s for up to 40 cycles.

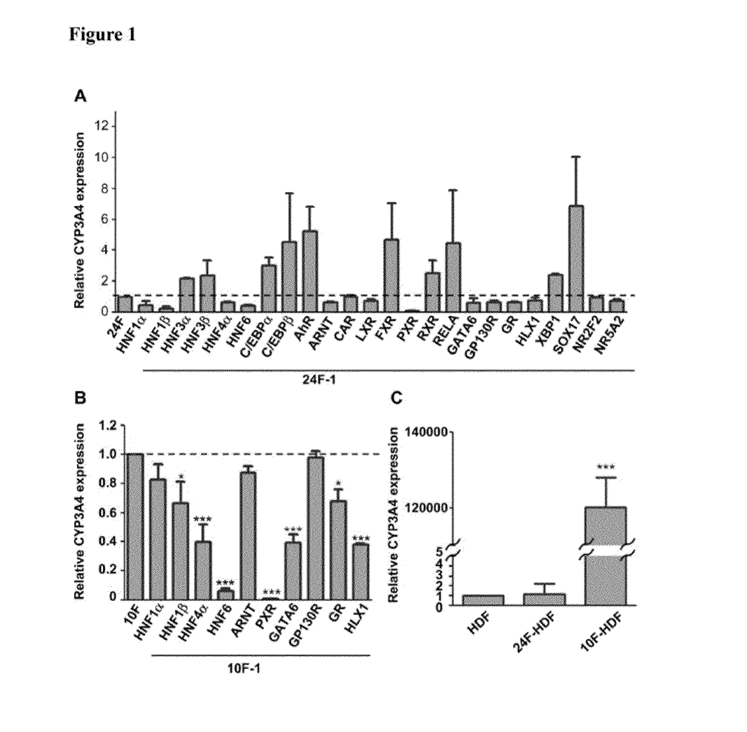

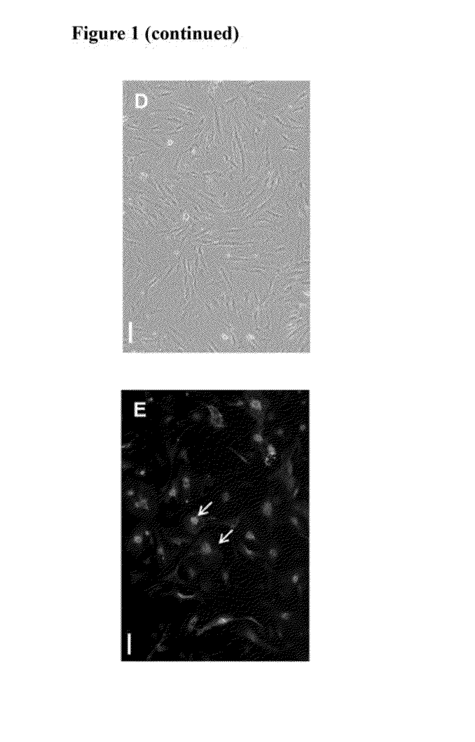

In FIG. 1A, the 1st bar indicated the expression level induced by the pool of 24 factors as the control. The later bars indicated the expression by the 23-factor pools omitting indicated factors, respectively, from the 24 factors. The Q-PCR analysis (FIG. 1A) showed omission of some factors increased CYP3A4 expression, while omission of others reduced the expression. The former factors were considered having inhibitory effects to CYP3A4 expression in HDFs and the latter ones having up-regulating effects. 10 from the latter factors were picked up for further studies. They included HNF1α, HNF 1β, HNF4α, HNF6, ARNT, PXR, GATA6, GP130R, GR and HLX1.

In FIG. 1B, the 1st bar indicated the CYP3A4 expression level by the pool of 10 factors selected from FIG. 1A as the control. The later bars indicated the expression level induced by the 9-factor pools omitting indicated factors, respectively, from the 10 factors. Elimination of anyone from the 10 did not increase the expression of CYP3A4 (FIG. 1B).

Compared to the original HDFs, transduction of 24 factors (24FHDFs) only increased the expression to 1.16-fold, while transduction of the selected 10 factors (10F-HDFs) dramatically increased it to 10⁵-fold (FIG. 1C). Compared to the original elongated HDFs, the 10F-HDFs became more ovoid in shape (arrows) and of course showed green fluorescence of the reporter copGFP (FIG. 1D, E).

### Example 2

### Comparing 10F-HDFs with Human Hepatoma Cell Lines

As alternatives to human hepatocytes whose sources were limited, human hepatoma cell lines were frequently used to determine drug metabolism though their drug-metabolizing activities were much lower than hepatocytes. Here, our 10F-HDFs were compared with human hepatoma cell line Hep3B, HepG2 and HuH-7 regarding their expression and functional activities of certain phase I enzymes CYP3A4, CYP1B1 and CYP2C9. Cell-based assays were used to determine cellular activities of CYP3A4, CYP2C9 and CYP1B1 (Promega Corp., Madison, Wis., USA). Cells were incubated with substrates (50 μM Luciferin-PFBE for CYP3A4, 100 μM Luciferin-H for CYP2C9, and 100 μM Luciferin-CEE for CYP1B1, respectively) at 37° C. for 3 h with occasional mixing by swirling or inverting. After incubation, an aliquot of 50 μL of medium was transferred from each well to a 96-well opaque white luminometer plate at room temperature, 50 μL of luciferin detection reagent was added into each well to initiate a luminescent reaction for 20 min in the dark, and then luminescence was read using Victor3 luminometer (PerkinElmer, Singapore). Following luminescence determinations, the cells were subjected to extraction of genomic DNA (Geneaid Biotech Ltd., Cleveland, Ohio, USA). The luminescence reads were normalized to the respectively yielded amount of genomic DNA. As usual, gene expression level determined by Q-PCR was normalized to their respective house-keeping gene GAPDH. For normalizing CYP activities, the yielded luminescence levels were divided by respective genomic DNA amount.

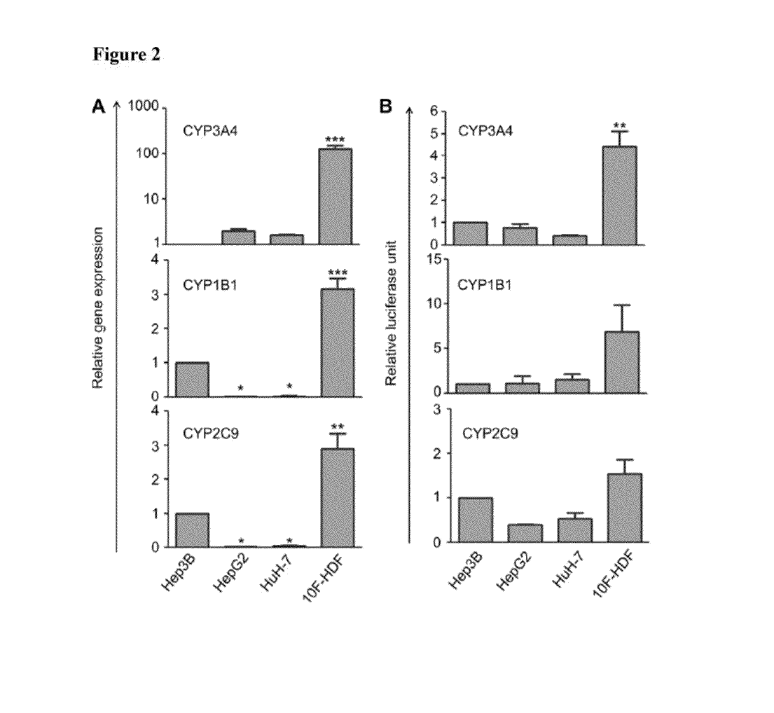

FIG. 2 showed remarkably higher levels of the expression (FIG. 2A) and activities (FIG. 2B) of the 3 CYPs in 10F-HDFs compared to the 3 hepatoma cell lines. Using Hep3B as the standard, 10F-HDFs were 103.9-fold higher on the expression and 4.4-fold higher on the functional activity of CYP3A4. The expression and activities of CYP I B I were 3.1- and 6.9-fold, and of CYP2C9 2.9- and 1.5-fold higher, respectively.

### Example 3

### Determination of Optimal Culture Conditions for Spheroid Formation of 10F-HDFs

Cells cultured into spheroids or into 3D scaffolds have been frequently reported to enhance or maintain longer their functions or phenotypes. Here it was tested whether 10F-HDFs cultured into spheroids and accommodated in certain scaffolds may enhance their CYP expression and activities.

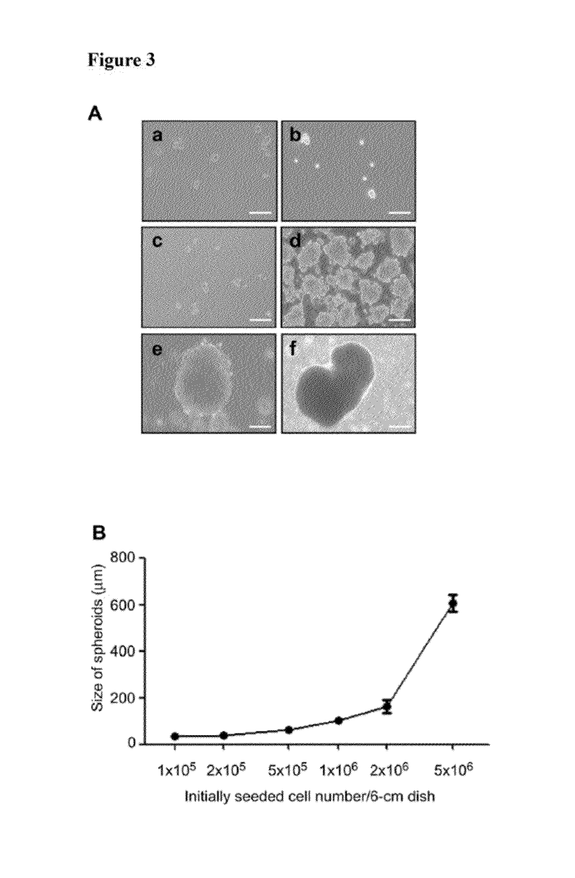

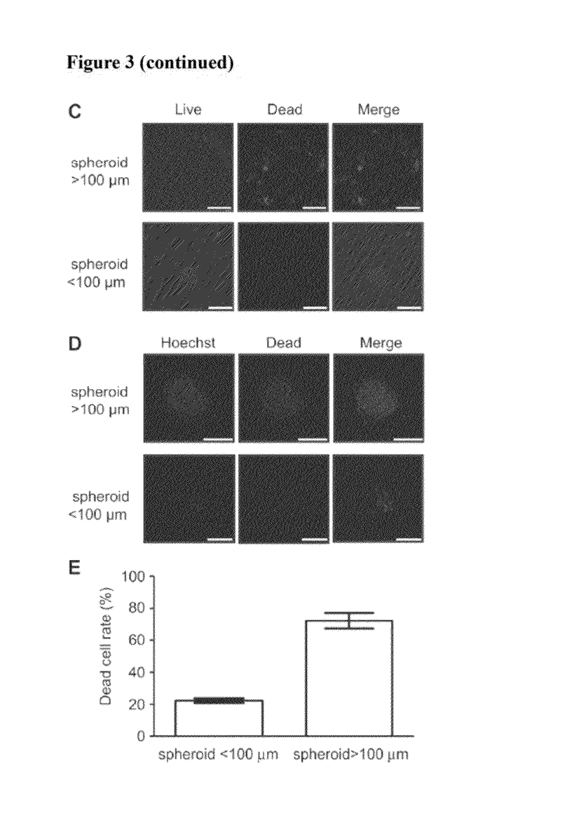

First, how big of the spheroids to be used must be decided. Using HydroCell dishes to form spheroids, the diameters of the formed spheroids were found closely correlated with the initial cell seeding densities. FIG. 3A showed the morphology of the spheroids formed on HydroCell dishes 4 days after culture of 10F-HDFs. The spheroids apparently became larger and larger as the initial cell seeding densities increased. After 4 days' culture, the average spheroid diameters were 35.3, 37.5, 63.8, 103.2, 163.5, and 606.7 μm, respectively, when cells were initially seeded at densities of 1×10⁵, 2×10⁵, 5×10⁵, 1×10⁶, 2×10⁶, and 5×10⁶ cells per 6-cm dish (surface area=21.5 cm²), respectively (FIG. 3B). Second, the dead cell rates in the variable-sized spheroids were determined. Cell viability in the spheroids was determined by LIVE/DEAD® Violet Viability/Vitality Kit (Invitrogen). Live cells were stained blue by calcein violet AM (ex/em=400/452±5 nm), dead cells stained red by Aqua-fluorescent reactive dye (ex/em=400/526±5 nm) and total cell nuclei were stained blue by Hoechst 34580 (ex/em=392/440±5 nm). The stained spheroids were imaged by a confocal microscope (TCS SP5 II, Leica, Buffalo Grove, Ill., USA). From the biggest plane of respective spheroids, dead cell number was divided by total nuclei number to yield a “dead cell rate”.

For this determination, the serial fluorescence images by the confocal microscopy of individual spheroids from their top to the bottom were acquired and the images at their largest diameter for measuring dead cell rates were selected. Representative fluorescence images of LIVE/DEAD and Hoechst/DEAD of spheroids larger or smaller than 100 μm were shown in FIGS. 3C and D, respectively. There was remarkably high dead cell rate (72%) in the spheroids larger than 100 μm, while much lower rate (22%) in those smaller than 100 μm (FIG. 3E). To minimize cell death possibly occurred in the spheroids during culturing; 10F-HDFs were therefore seeded on the 6-cm HydroCell dishes at a starting density of 1×10⁶ cells/dish in the following experiments. The spheroids thus formed had an average diameter of approximately 100 μm after 4 days of culture.

### Example 4

### Enhancement of CYP3A4 Expression in 10F-HDFs Cultured in Various Kinds of Scaffolds

10F-HDFs in single cell suspension were seeded into various kinds of scaffolds and cultured for 10 days to examine which scaffold would enhance the expression of CYP3A4 in 10F-HDFs to the utmost. The scaffold cubes were sterilized with 750% ethanol and then washed twice with phosphate-buffered saline (PBS) to remove the residual ethanol before cell seeding. The cells in single cell suspension or in spheroids were suspended in medium and then seeded into scaffolds by gravity. The scaffolds were placed in a culture dish for 30 min for cell adhesion, and then cultured in 6-well plates for 7 days before assessment of gene expression and enzyme activities. The medium was changed every other day.

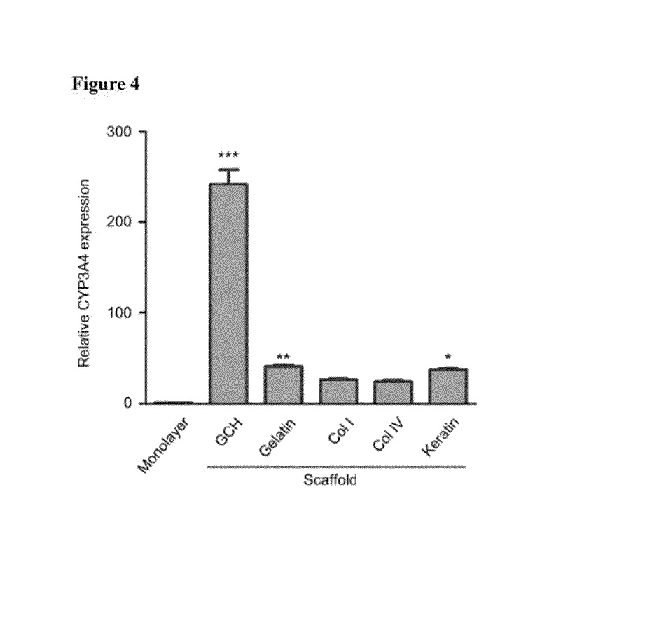

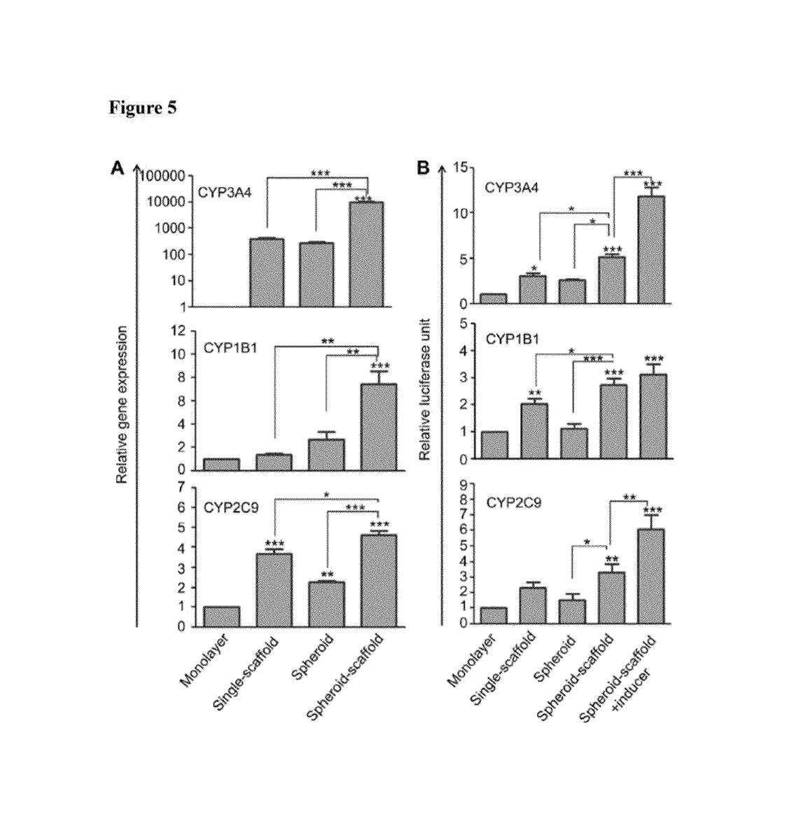

Compared to 10F-HDFs cultured in monolayer, expression levels of CYP3A4 in 10F-HDFs increased to 40-fold, 26-fold, 24-fold or 37-fold when the cells were cultured in gelatin, type I collagen, type IV collagen or keratin scaffold, respectively (FIG. 4). More strikingly, the expression increased to as high as 242-fold (FIG. 4) to 360-fold (FIG. 5A) when 10F-HDFs were cultured in GCH tri-copolymer scaffold. Thus GCH scaffold was chosen for further experiments.

### Example 5

### Further Enhancement of the Gene Expression and Enzyme Activities of CYPs in 10F-HDFs Cultured as Spheroids and Seeded Inside the GCH Tri-Copolymer Scaffold

Next, it was examined whether spheroid culture of 10F-HDFs in GCH tri-copolymer scaffold could further enhance the expression and activities of CYPs including CYP3A4. To compare with 10F-HDFs cultured in monolayer, the cells in single suspension were directly seeded into GCH scaffold (single-scaffold), cultured into spheroids on HydroCell dishes (spheroid), or the formed spheroids were further seeded and cultured in the GCH scaffolds (spheroid-scaffold) for 1 week before determination of gene expression and enzyme activities.

Similarly to FIG. 4, FIG. 5A shows remarkable enhancement of CYP3A4 expression in single-scaffold culture (360-fold). This culture condition could not increase the expression of CYP1B1 but slightly increased the expression of CYP2C9 to 3.7-fold. Spheroid culture also markedly increased the expression of CYP3A4 (260-fold), but only slightly increased the expression of CYP1B1 (2.7-fold) and CYP2C9 (2.3-fold). Furthermore, spheroid-scaffold culture condition more significantly increased the expression of CYP3A4 to 9760-fold. Expression of CYP 1B1 and CYP2C9 were increased to 7.4- and 4.6-fold, respectively.

The enzyme activities which were normalized to respective genomic DNA amount were shown in FIG. 5B. Spheroid-scaffold culture again significantly increased the activities of the 3 CYPs (CYP3A4, 5.2-fold; CYP1B1, 2.7-fold; CYP2C9, 3.3-fold). Single scaffold culture slightly increased the activities of CYP3A4 (3-fold), CYP1B1 (2.0-fold) and CYP2C9 (2.3-fold). Spheroid culture only mildly increased the activities of CYP3A4 (2.6-fold) but did not influence the activities of CYP1B1 (1.1-fold) and CYP2C9 (1.5-fold). Addition of rifampicin (inducer of CYP3A and CYP2C) to the spheroid-scaffold culture obviously induced enzyme activities of CYP3A4 (2.3-fold) and CYP2C9 (1.8-fold). However, omeprazole (inducer of CYP I B I) did not induce CYP1B1 activities in the spheroid-scaffold culture.

### Example 6

### Demonstration of 10F-HDF Spheroids Accommodated within the GCH Tri-Copolymer Scaffold

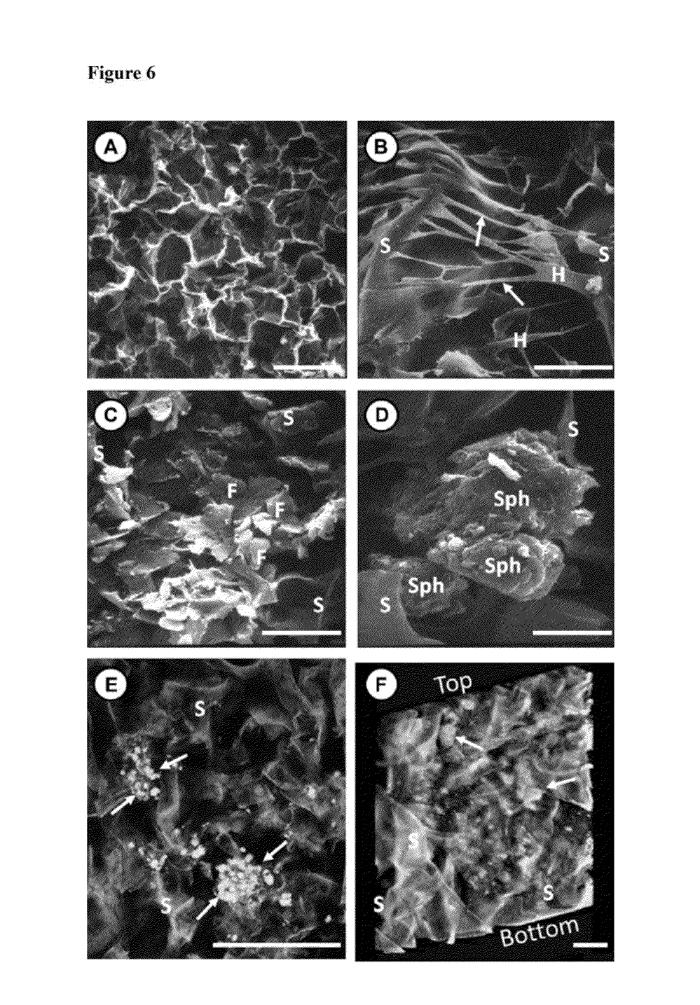

An empty scaffold imaged by SEM was shown in FIG. 6A. The pores were highly interconnected as a network which is important for cell infiltration and nutrient transportation. After cultured for 7 days, the scaffolds containing cells were dehydrated by treatment with a series of graded ethanol solutions and followed by critical point drying. They were subsequently coated with gold for imaging by scanning electron microscopy (SEM, S-800 Field emission scanning electron microscope, Hitachi, Tokyo, Japan). HDFs (FIG. 6B) and 10F-HDFs (FIG. 6C) seeded as single cell suspension in the scaffold demonstrated remarkable difference in cell morphology. The HDFs were typically fibroblastic with elongated cytoplasmic extensions, while 10F-HDFs were more or less tightly packed and with much less extensions. FIG. 6D shows irregularly shaped spheroids lodged inside the pores of the scaffold.

As there is reporter copGFP expressed by the recombinant lentiviruses, the virus-infected 10F-HDFs were readily detectable by the confocal microscopy and shown green. FIG. 6E shows cells in spheroids, small cell clusters, and as single cells inside the scaffold. A representative optical section of reconstructed microscopic images (FIG. 6F) showed scattered distribution of the spheroids and small cell clusters along the vertical plane. The larger sized spheroids tended to be lodged in the upper part of the scaffold as they were seeded by gravity in this case.

### Example 7

### Persistence of Enhanced CYP3A4 Expression and Activities in the Spheroid-Scaffold Cultures for 2 Weeks

The spheroid-scaffold constructs were cultured for 1-4 weeks to follow up their duration of enhanced CYP3A4 expression and activities. A stock solution of oxidized nifedipine (ONIF, Sigma) at 100 nmol/mL was prepared with acetonitrile (ACN)-water solution (80:20, v/v). A standard calibration curve of ONIF was constructed with concentrations of 0.001, 0.01, 0.05, 0.1, 0.5, 1, and 10 nmol/mL. To measure cellular activities of CYP3A4 that metabolize NIF into ONIF, cells were incubated in the medium containing 10 mg/mL NIF for 24 h. An aliquot of 500 μL of the medium was centrifuged at 12,000×g for 10 min and then the supernatant was mixed with the same volume of 80% ACN before measurement of ONIF by high performance liquid chromatography-electrospray tandem mass spectrometry (HPLCeESIeMS/MS). A ThermoFinnigan LXQ Advantage ion trap mass spectrometer (San Jose, Calif., USA) coupled with a ThermoFinnigan Surveyor liquid chromatography system was employed for the measuring. The extracts were injected into a reversed phase column (Phenomenex Kinetex 2.6 u, C18, 100A, 150×2.10 mm) at a flow rate of 0.2 mL/min. Eluted peaks at 269 nm for ONIF were monitored by a photodiode array detector. The area under each peak was recorded and interpolated using the standard line to indicate moles of ONIF, and then normalized by respective amount of genomic DNA.

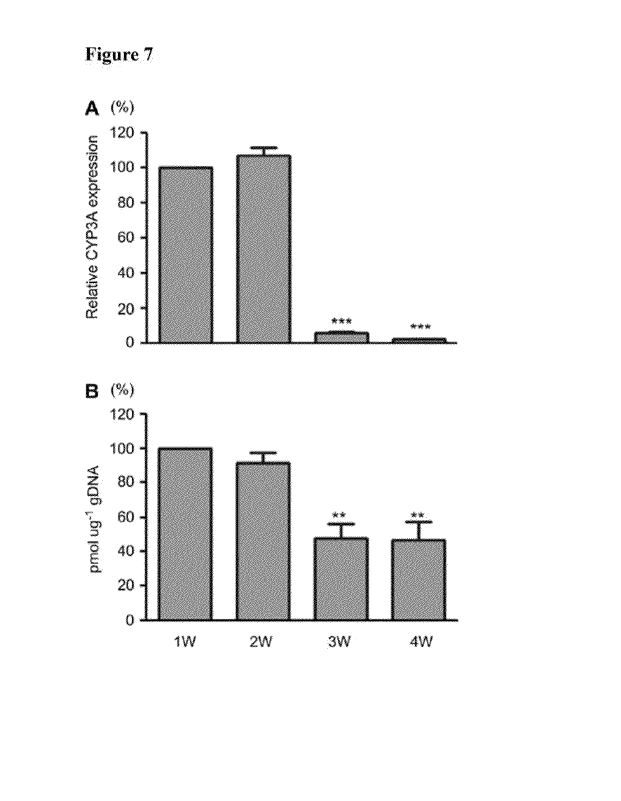

By HPLC-ESI-MS/MS, the CYP3A4 activities were here determined by measuring the amount of ONIF after incubating the constructs for 24 h with 10 μg/mL NIF. The amount of ONIF was then normalized to respective genomic DNA amount. FIG. 7A shows CYP3A4 expression determined by Q-PCR was dramatically decreased at 3 weeks, while its activities (FIG. 7B) was highest at 1 week, and started to decline significantly since 3 weeks.

### Example 8

### The Gene Expression and Enzyme Activities of CYPs in HDFs, MSCs and ADSCs

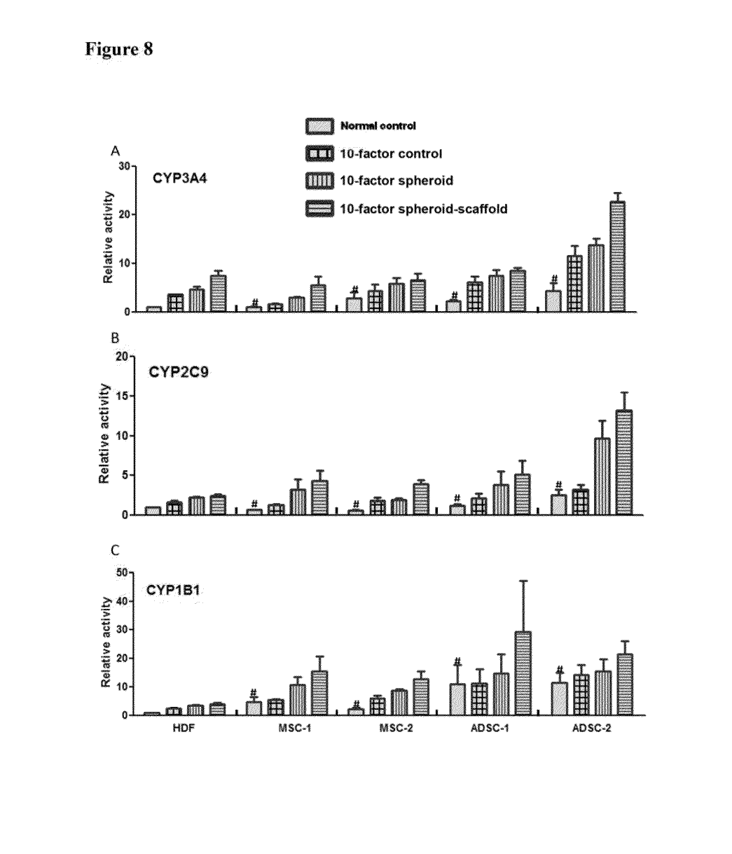

In FIG. 8, the basal levels (normal controls without addition of 10 factors) of CYP3A4, CYP2C9 and CYP1B1 activities were significantly higher in MSCs and ADSCs than in HDF. Compared with respective normal controls, induction folds by 10-factor delivery (respective 10-factor controls) in HDFs, MSC1, MSC2, ADSC1, and ADSC2 for CYP3A4 were 3.52-, 1.68-, 1.61-, 3-, and 2.94-fold, respectively; for CYP2C9 1.57-, 2.01-, 3-, 1.78-, and 1.3-fold, respectively; and for CYP1B1 2.51-, 2.06-, 3.35-, 1.22-, and 1.27-fold, respectively. Using normal control of HDFs as the standard (=1), stepwise increases of CYP3A4 activities in HDFs were found from 3.52-fold higher in 10-factor control culture, 4.62-fold in spheroid culture and 7.48-fold in spheroid-scaffold culture. Similar stepwise increases were also shown in CYP2C9 and CYP1B1 in HDFs, and also in all the 3 CYPs in 2 MSCs and 2 ADSCs. However, ADSCs showed more significant increases of CYP3A4 activities than HDFs and MSCs in the 3D culture conditions (spheroid 7.4- and 13.76-fold and spheroid-scaffold 8.49- and 22.68-fold in ADSC1 and ADSC2, respectively, vs. spheroid 3- and 5.81-fold and spheroid-scaffold 5.57- and 6.5-fold in MSC I and MSC2, respectively, vs. spheroid 4.62- and spheroid-scaffold 7.48-fold in HDFs). The increases of CYP2C9 activities in ADSCs (spheroid 3.8- and 9.7-fold and spheroid-scaffold 5.08- and 13.12-fold, respectively, for ADSC1 and ADSC2) were more significant than in MSCs (spheroid 3.18- and 1.86-fold and spheroid-scaffold 4.29- and 3.90-fold, respectively, for MSC1 and MSC2). Similarly, the increases of CYP1B1 activities in 3D culture conditions were also more striking in ADSCs than in MSCs (spheroid 14.73- and 15.33-fold and spheroid-scaffold 29.25- and 21.35-fold, respectively, for ADSC and ADSC2 vs. spheroid 10.78- and 8.70-fold and spheroid-scaffold 15.35- and 12.72-fold, respectively, for MSC and MSC2). In conclusion, regarding the most powerful CYP induction condition, the spheroid-scaffold culturing, ADSCs were more easily enhanced than MSCs and HDFs.

1. A kit, comprising:
(a) a cell transfected with hepatic transcription regulators; and
(b) a culture medium that support growth of the cell,
wherein the culture medium is a 2-dimensional culture medium or a 3-dimensional scaffold, wherein the cell is a human dermal fibroblast, a mesenchymal stem cell, or an adipose-derived stem cell.

2. (canceled)

3. The kit of claim 1, wherein the 3-dimensional scaffold is a gelatin scaffold, a type I collagen scaffold, a type IV collagen scaffold, a keratin scaffold, or a gelatin-chondroitin-hyaluronan tri-copolymer scaffold.

4. The kit of claim 1, wherein the cell forms a monolayer or a spheroid on the culture medium.

5. The kit of claim 1, wherein the cell is induced to express cytochrome P450 (CYP) enzymes by the hepatic transcription regulators.

6. The kit of claim 5, wherein the cytochrome P450 is CYP3A4, CYP1B1 or CYP2C9.

7. A method for determining a drug metabolism, comprising:
(a) transfecting a cell with hepatic transcription regulators;
(b) culturing the cell on a medium;
(c) adding the drug into the medium; and
(d) measuring a concentration change of the drug,
wherein the cell is a human dermal fibroblast, a mesenchymal stem cell, or an adipose-derived stem cell.

8. (canceled)

9. The method of claim 7, wherein the medium is a 2-dimensional culture medium or a 3-dimensional scaffold.

10. The method of claim 9, wherein the 3-dimensional scaffold is a gelatin scaffold, a type I collagen scaffold, a type IV collagen scaffold, a keratin scaffold, or a gelatin-chondroitin-hyaluronan tri-copolymer scaffold.

11. The method of claim 9, wherein the cell forms a monolayer or a spheroid on the medium.

12. The method of claim 7, wherein the cell is induced to express cytochrome P450 (CYP) enzymes by the hepatic transcription regulators.

13. The method of claim 12, wherein the cytochrome P450 is CYP3A4, CYP1B1 or CYP2C9.

14. A method for predicting a drug toxicity, comprising:
(a) transfecting a cell with hepatic transcription regulators;
(b) culturing the cell on a medium;
(c) adding the drug into the medium; and
(d) measuring a death rate of the cell,
wherein the cell is a human dermal fibroblast, a mesenchymal stem cell, or an adipose-derived stem cell.

15. (canceled)

16. The method of claim 14, wherein the medium is a 2-dimensional culture medium or a 3-dimensional scaffold.

17. The method of claim 16, wherein the 3-dimensional scaffold is a gelatin scaffold, a type I collagen scaffold, a type IV collagen scaffold, a keratin scaffold, or a gelatin-chondroitin-hyaluronan tri-copolymer scaffold.

18. The method of claim 16, wherein the cell forms a monolayer or a spheroid on the medium.

19. The method of claim 14, wherein the cell is induced to express cytochrome P450 (CYP) enzymes by the hepatic transcription regulators.

20. The method of claim 19, wherein the cytochrome P450 is CYP3A4, CYP1B1 or CYP2C9.
